(19) United States
(12) Patent Application Publication
(10) Pub. No.: US 2023/0003740 A1
(43) Pub. Date: Jan. 5, 2023

(54) METHOD FOR DIAGNOSING ESTHETIC DEGRADATIONS OF SKIN

(71) Applicant: L'OREAL, Paris (FR)

(72) Inventors: Namita MISRA, Aulnay-Sous-Bois (FR); Nükhet CAVUSOGLU, Aulnay-Sous-Bois (FR); Cécile CLAVAUD, Aulnay-Sous-Bois (FR)

(21) Appl. No.: 17/621,382

(22) PCT Filed: Jun. 24, 2020
(86) PCT No.: PCT/EP2020/067627

§ 371 Date: Dec. 21, 2021

(30) Foreign Application Priority Data
Jun. 24, 2019 (FR) 1906831

(51) Int. Cl. G01N 33/68; A61B 5/00; C12Q 1/14; C12Q 1/689

(52) U.S. Cl. CPC G01N 33/6881; A61B 5/442; C12Q 1/14; C12Q 1/689; G01N 33/6848; G01N 2800/207

(57) ABSTRACT

The present invention relates to a method for diagnosing esthetic degradations of skin, in particular linked to pollution, in a subject, comprising a step (a) of determining, in a skin sample of the subject, the level of at least one marker chosen from the group constituted of (i) bacteria of the species Propionibacterim acnes, bacteria of the family Micrococcaceae, bacteria of the genus Brachybacterium, bacteria of the genus Brevibacterium, bacteria of the order Burkholderiales, bacteria of the genus Parococcus, bacteria of the family Rhodobacteraceae and bacteria of the genus Fusobacterium, and (ii) metabolites of these bacteria chosen from 3-hydroxy-3-methylglutarate, 3-methylglutarate/2-methylglutarate, 4-guanidinobutanoate, 4-imidazoleacetate, 5-oxoproline, aconitrate, adipate, alanine, alpha-cetoglutarate, arabonate/xylonate, azelate, beta-citrylglutamate, choline, cis-urocanate, citraconate/glutaconate, fructose, fumarate, gamma-glutamylalanine, gamma-glutamylglutamine, gamma-glutamylglycine, gamma-glutamylisoleucine, gamma-glutamylleucine, gamma-glutamylsérine, gamma-glutamylthréonine, gamma-glutamyltryptophane, gamma-glutamylvaline, glutarate, glycerate, glycerol-3-phosphate, glycine, isovalerylglycine, kynurenate, lactate, linoleoyl ethanolamide, malate, maleate, malonate, maltose, methionine sulfoxide, methylsuccinate, N-acetylalanine, N-acetylarginine, N-acetylaspartate, N-acetylglycine, N-acetylhistidine, N-acetylphenylalanine, N-acetylthréonine, N-acetylvaline, oleamide, ornithine, palmitamide, pimelate, proline, salicylate, sebacate, serine, suberate, succinate, undecanedioate and S-amino-omega caprolactam.

The present invention relates to the diagnosing of esthetic degradations of skin, in particular linked to pollution.

Many factors can generate imperfections on the skin and its complexion. These imperfections of the complexion of the skin are a more and more frequent cause of consultations at esthetic care centers or dermatologists' offices.

Among the extrinsic factors likely to affect the complexion of the skin, mention can be made of exposure to the sun, exposure to variations in temperature and/or humidity, exposure to pollutants or to cigarette smoke. Among the intrinsic factors that affect the complexion of the skin, mention can be made of stress, fatigue, hormonal changes, dehydration of the epidermis, an alternation in the barrier function of the skin, aging or excessive secretion of sebum.

These different factors tent to blur the complexion, make it inhomogeneous, dull, waxy or yellowish and cause, and even favor, the presence of pigmentary disorders.

Yet, to prevent and/or treat these esthetic degradations of skin, it can be useful to know the cause of it, in particular from among the factors mentioned hereinabove.

Certain urban environments are regularly subjected to pollution peaks. The individual in his daily environment and particularly in an urban zone, can be subjected to multiple aggressions at the keratin materials, and in particular the skin, by different air pollutants.

Among the known pollutants, mention can first be made of exhaust gas which has become a major challenge in big cities, heavy metals but also fine particles or polycyclic aromatic hydrocarbons such as benzopyrene or benzoanthracene.

These pollutants will notably cause, on the skin, the deposition of particles on the surface of the epidermis, and among other consequences, induce a dull complexion of the skin and/or pigmentary disorders.

Identifying the exposure to pollution as a factor responsible for esthetic degradations of skin could make it possible to prevent and/or treat these degradations more effectively, There is therefore a substantial need for methods for diagnosing esthetic degradations of skin, in particular linked to pollution.

This invention meets this need.

The present invention arises from the unexpected finding by the inventors that skin samples of individuals exposed to chronic pollution (based on the detection of high levels of pollutants in the samples of their hair) displayed a level of certain bacteria and of certain metabolites of these bacteria that was significantly higher with respect to individuals not exposed to such pollution.

The present invention thus relates to a method for diagnosing esthetic degradations of skin, in particular linked to pollution, in a subject, comprising a step (a) of determining, in a skin sample of the subject, the level of at least one marker selected from the group constituted of (i) bacteria that comprise a nucleic acid encoding a 16S rRNA of sequence at least 90% identical to the sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9, and (ii) metabolites of these bacteria selected from 3-hydroxy-3-methylglutarate, 3-methylglutarate/2-methylglutarate, 4-guanidinobutanoate, 4-imidazoleacetate, 5-oxoproline, aconitrate, adipate, alanine, alpha-cetoglutarate, arabonate/xylonate, azelate, beta-citrylglutamate, choline, cis-urocanate, citraconate/glutaconate, fructose, fumarate, gamma-glutamylalanine, gamma-glutamylglutamine, gamma-glutamylglycine, gamma-glutamylisoleucine, gamma-glutamylleucine, gamma-glutamylserine, gamma-glutamylthreonine, gamma-glutamyltryptophane, gamma-glutamylvaline, glutarate, glycerate, glycerol-3-phosphate, glycine, isovalerylglycine, kynurenate, lactate, linoleoyl ethanolamide, malate, maleate, malonate, maltose, methionine sulfoxide, methylsuccinate, N-acetylarginine, N-acetylalanine, N-acetylaspartate, N-acetylglycine, N-acetylhistidine, N-acetylphenylalanine, N-acetylthreonine, N-acetylvaline, oleamide, ornithine, palmitamide, pimelate, proline, salicylate, sebacate, serine, suberate, succinate, undecanedioate and S-amino-omega caprolactam.

In a particular embodiment, said at least one marker is selected from the group constituted as of (i) bacteria of the species Propionibacterim acnes, bacteria of the family Micrococcaceae, bacteria of the genus Brachybacterium, bacteria of the genus Brevibacterium, bacteria of the order Burkholderiales, bacteria of the genus Parococcus, bacteria of the family Rhodobacteraceae and bacteria of the genus Fusobacterium, and (ii) metabolites of these bacteria selected from 3-hydroxy-3-methylglutarate, 3-methylglutarate/2-methylglutarate, 4-guanidinobutanoate, 4-imidazoleacetate, 5-oxoproline, aconitrate, adipate, alanine, alpha-cetoglutarate, arabonate/xylonate, azelate, beta-citrylglutamate, choline, cis-urocanate, citraconate/glutaconate, fructose, fumarate, gamma-glutamylalanine, gamma-glutamylglutamine, gamma-glutamylglycine, gamma-glutamylisoleucine, gamma-glutamylleucine, gamma-glutamylserine, gamma-glutamylthreonine, gamma-glutamyltryptophane, gamma-glutamylvaline, glutarate, glycerate, glycerol-3-phosphate, glycine, isovalerylglycine, kynurenate, lactate, linoleoyl ethanolamide, malate, maleate, malonate, maltose, methionine sulfoxide, methylsuccinate, N-acetylarginine, N-acetylalanine, N-acetylaspartate, N-acetylglycine, N-acetylhistidine, N-acetylphenylalanine, N-acetylthreonine, N-acetylvaline, oleamide, ornithine, palmitamide, pimelate, proline, salicylate, sebacate, serine, suberate, succinate, undecanedioate and S-amino-omega caprolactame.

In another particular embodiment, said at least one marker is chosen from the group constituted of (i) bacteria of the species Micrococcus luteus and bacteria of the species Paracoccus sp., and (ii) metabolites of these bacteria chosen from kynurenate, 4-imidazoleacetate, maleate, ornithine, 4-guanidinobutanoate, cis-urocanate, malonate, gamma-glutamylleucine, N-acetylarginine and glycerol-3-phosphate.

Another object of the invention relates to a method for evaluating the cutaneous exposure of a subject to pollution, comprising a step (a) of determining, in a skin sample of the subject, the level of at least one marker selected from the group constituted of (i) bacteria that comprise a nucleic acid encoding a 16S rRNA of a sequence at least 90% identical to the sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9, and (ii) metabolites of these bacteria chosen from 3-hydroxy-3-methylglutarate, 3-methylglutarate/2-methylglutarate, 4-guanidinobutanoate, 4-imidazoleacetate, 5-oxoproline, aconitrate, adipate, alanine, alpha-cetoglutarate, arabonate/xylonate, azelate, beta-citrylglutamate, choline, cis-urocanate, citraconate/glutaconate, fructose, fumarate, gamma-glutamylalanine, gamma-glutamylglutamine, gamma-glutamylglycine, gamma-glutamylisoleucine, gamma-glutamylleucine, gamma-glutamylserine, gamma-glutamylthreonine, gamma-glutamyltryptophane, gamma-glutamylvaline, glutarate, glycerate, glycerol-3-phosphate, glycine, isovalerylglycine, kynurenate, lactate, linoleoyl ethanolamide, malate, maleate, malonate, maltose, methionine sulfoxide, methylsuccinate, N-acetylarginine, N-acetylalanine, N-acetylaspartate, N-acetylglycine, N-acetylhistidine, N-acetylphenylalanine, N-acetylthreonine, N-acetylvaline, oleamide, ornithine, palmitamide, pimelate, proline, salicylate, sebacate, serine, suberate, succinate, undecanedioate and S-amino-omega caprolactam.

### DETAILED DESCRIPTION OF THE INVENTION

Marker Microbiome

The marker used in the context of the invention can be chosen from the group constituted of bacteria comprising a nucleic acid encoding a 16S rRNA of a sequence at least 90% identical to the sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9.

“16S rRNA” here means the ribosomal RNA that form the small subunit of ribosomes of prokaryotes. The genes encoding 16S rRNA are called “16S rDNA”. Their sequence is highly used due to its structure, well conserved in all bacteria. Indeed, it is constituted of a succession of conserved domains, complementarity regions for universal primers used for the sequencing of this gene, and other sequence portions specific to a group of bacteria, named signature sequences (species, genus, family, order).

Thus, the marker used in the context of the invention can be chosen from the group constituted of bacteria comprising a 16S rDNA of sequence at least 90% identical to the sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9.

In the context of the present invention, the “percentage identity” is calculated by using a global alignment (i.e. the two sequences are compared over their entire sequence). Methods for comparing the identity of two sequences or more are well known to those skilled in the art. The “needle” program, which uses the Needleman-Wunsch global alignment 90 algorithm (Needleman and Wunsch (1970) J. Mol. Biol. 48:443-453) to find the optimum alignment (including the gaps) between two sequences by considering their entire sequence can for example be used. The needle program is for example available on the internet site ebi.ac.uk. The percentage identity according to the invention is preferably calculated by using the needle program (global) EMBOSS with a “Gap Open” parameter equal to 10.0, a as “Gap Extend” parameter equal to 0.5, and a DNAFULL matrix.

In a particular embodiment, the marker used in the context of the invention can be chosen from the group constituted of bacteria comprising a nucleic acid encoding a 16S rRNA, in particular a 16S rDNA, of sequence at least 91% identical, in particular at least 92%, at least 93%, at least 94%, at least 95%, at least 96%, at least 98%, at least 99%, at least 99,5%, at least 99,9% or at least 100% identical, to the sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9.

The sequence SEQ ID NO: 1 is a representative sequence of 16S rDNA of bacteria of the species Propionibacterium acnes.

The sequence SEQ ID NO: 2 is a representative sequence of 16S rDNA of bacteria of the family Micrococcaceae.

The sequence SEQ ID NO: 3 is a representative sequence of 16S rDNA of bacteria of the genus Brachybacterium.

The sequence SEQ ID NO: 4 is a representative sequence of 16S rDNA of bacteria of the genus Brevibacterium.

The sequence SEQ ID NO: 5 is a representative sequence of 16S rDNA of bacteria of the order Burkholderiales.

The sequence SEQ ID NO: 6 is a representative sequence of 16S rDNA of bacteria of the genus Parococcus.

The sequence SEQ ID NO: 7 is a representative sequence of 16S rDNA of bacteria of the family Rhodobacteraceae.

The sequence SEQ ID NO: 8 is a representative sequence of 16S rDNA of bacteria of the genus Fusobacterium.

The sequence SEQ ID NO: 9 is a representative sequence of 16S rDNA of bacteria of the species Micrococcus luteus.

Thus, in a particular embodiment, the marker used in the context of the invention is chosen from the group constituted of bacteria of the species Propionibacterium acnes, bacteria of the family Micrococcaceae, bacteria of the genus Brachybacterium, bacteria of the genus Brevibacterium, bacteria of the order Burkholderiales, bacteria of the genus Parococcus, bacteria of the family Rhodobacteraceae and bacteria of the genus Fusobacterium,

“Bacterium of the species Propionibacterium acnes” or Cutibacterium acnes, here means a Gram positive bacterium, anaerobic and saprophytic, bacilliform linked to acne, of the order Actinomycetales, of the family Propionibacteriaceae and of the genus Propionibacterium.

“Bacterium of the family Micrococcaceae” here means a spherical Gram positive coccus and in cluster, catalase+. The family Micrococcaceae includes the genera Acaricomes, Arthrobacter, Auridibacter, Citricoccus, Enteroactinococcus, Kocuria, Micrococcus, Nesterenkonia, Renibacterium, Rothia, Sinomonas, Tersicoccus, Yaniella and Zhihengliuella.

In a particular embodiment, the bacterium of the family Micrococcaceae is a bacterium of the genus Micrococcus.

“Bacterium of the genus Micrococcus” here means a Gram positive bacterium, from 0.5 to 2 μm in diameter, often grouped in tetrads or irregular clusters, aerobic, with oxidative metabolism, having a catalase and chemoorganotrophic.

The genus Micrococcus includes the species Micrococcus luteus, Micrococcus lylae, Micrococcus varians, Micrococcus roseus, Micrococcus agilis, Micrococcus kristinae, Micrococcus nishinomiyaensis, Micrococcus sedentarius, Micrococcus halobius, Micrococcus antarticus, Micrococcus cohnii, Micrococcus endophyticus, Micrococcus flavus, Micrococcus lactis, Micrococcus terreus and Micrococcus yunnanensis.

In a particular embodiment, the bacterium of the family Micrococcaceae is a bacterium of the species Micrococcus luteus.

“Bacterium of the genus Brachybacterium” here means a non-mobile Gram-positive bacterium, of the family Dermabacteraceae, of which the typical species is Brachybacterium faecium.

“Bacterium of the genus Brevibacterium” here means a Gram-positive bacterium of the order Actinomycetales and of the family Brevibacteriaceae.

“Bacterium of the order Burkholderiales” here means a Gram-negative proteobacterium. The order Burkholderiales includes the family Alcaligenaceae, the family Burkholderiaceae, the family Comamonadadeae, the family Oxalobacteraceae and the family Sutterellaceae.

“Bacterium of the family Rhodobacteraceae” here means a Gram-negative proteobacterium of the order Rhodobacterales.

The family Rhodobacteraceae includes the genera Actibacterium, Acuticoccus, Aestuariibius, Aestuariicoccus, Aestuariivita, Agaricicola, Ahrensia, Albidovulum, Albimonas, Albirhodobacter, Aliiroseovarius, Aliisedimentitalea, Alkalimicrobium, Amaricoccus, Amylibacter, Antarctobacter, Aquicoccus, Aquimixticola, Asc/diaceihabitans, Boseongicola, Brevirhabdus, Catellibacterium, Celeribacer, Cereibacer, Citreicella, Citreimones, Cognatishimia, Cognafiyoonia, Confluentimicrobium, Cribrihabitans, Defluviimonas, Dinoroseobacter, Donghicola, Epibacterium, Falsirhodobacter, Flavimaricola, Frigidibacter, Gaetbulicola, Gemmobacter, Haematobacter, Halocynthiibacter, Halodurantibacterium, Halovulum, Hasllibacter, Hirschia, Huaishuia, Hwanghaeicola, Hyphomonas, Jannaschia, Jhaorihella, Kandellimicrobium, Ketogulonicigenium, Labrenzia, Lacimonas, Leisingera, Lentibacter, Limibaculum, Limimaricola, Litoreibacter, Litorimicrobium, Litorisediminicola, Litorisediminivivens, Litorivivens, Lokanella, Lutimaribacter, Maliponia, Mameliella, Mangrovicoccus, Maribius, Maricaulis, Marimonas, Marinibacterium, Marinovum, Maritirnibacter, Marivita, Marivivens, Methylarcula, Monaibacterium, Nautella, Neptunicoccus, Nereida, Nesiotobacter, Nioella, Nitropelagi, Oceanibulbus, Oceanicaulis, Oceanicella, Oceanicola, Oceaniglobus, Oceaniovalibus, Octadecabacter, Pacificibacter, Paenirhodobacter, Palleronia, Pannonibacter, Paracoccus, Paradonghicola, Paraphaeobacer, Pararhodobacter, Pelagibeca, Pelagicola, Pelagimonas, Phaeobacter, Planktomarina, Planktotalea, Plastorhodobacter, Pleomorphobacterium, Polymorphum, Pontibaca, Ponticoccus, Pontivivens, Primorskyibacter, Profundibacterium, Pseudaestuariivita, Pseudodonghicola, Pseudohalocynthiibacter, Pseudomaribius, Pseudooceanicola, Pseudooctadecabacter, Pseudopelagicola, Pseudophaeobacter, Pseudorhodobacter, Pseudoroseicyclus, Pseudoroseovarius, Pseudoruegeria, Pseudoseohaeicola, Pseudovibrio, Psychromarinibacter, Puniceibacterium, Rhodobaca, Rhodobaculum, Rhodosalinus, Rhodovulum, Roseibaca, Roseibacterium, Roseibacula, Roseibium, Roseicitreum, Roseicyclus, Roseinatronobacter, Roseisalinus, Roseivivax, Roseobacter, Roseovarius, Rubellimicrobium, Rubribacterium, Rubricalla, Rubrimonas, Ruegeria, Sagittula, Salinihabitans, Salinovum, Salipiger, Sedimentitalea, Sediminimonas, Seohaeicola, Shimia, Silicibacter, Silicimonas, Sinorhodobacter, Staleya, Stappia, Sulfitobacter, Tabrizicola, Tateyamaria, Thalassobacter, Thalassobius, Thalassococcus, Thiobacimonas, Thioclava, Thiosphaera, Tranquillimonas, Tritonibecter, Tropicibacter, Tropicimonas, Vadicella, Wenxinia, Xinfangfangia, Xuhuaishuia, Yangia, Yoonia and Youngimonas. “Bacterium of the genus Paracoccus” here means a Gram-negative bacterium of the family Rhodobacteraceae, of which the typical species is Paracoccus dendrificans. The genus Paracoccus includes the species Paracoccus acridae, Paracoccus aerius, Paracoccus aestuarii. Paracoccus aestuarivivens, Paracoccus alcaliphilus, Paracoccus alimentarius, Paracoccus alkenifer, Paracoccus aminophilus, Paracoccus aminovorans, Paracoccus angustae, Paracoccus bengalensis, Paracoccus caeni, Paracoccus carotinifaciens, Paracoccus cavernae, Paracoccus chinensis, Paracoccus communis, Paracoccus contaminans, Paracoccus denitrificans, Paracoccus fistulariae, Paracoccus fontiphilus, Paracoccus haeundaensis, Paracoccus halodenitrificans, Paracoccus halophilus, Paracoccus hibisci, Paracoccus hibiscisoli, Paracoccus homiensis, Paracoccus huijuniae, Paracoccus isoporae, Paracoccus kocurii, Paracoccus kondratievae, Paracoccus koreensis, Paracoccus laeviglucosivorans, Paracoccus limosus, Paracoccus lilorisediminis, Paracoccus lutimaris, Paracoccus mangrovi, Paracoccus marcusii, Paracoccus marinus, Paracoccus methylutens, Paracoccus niistensis, Paracoccus pacificus, Paracoccus panacisoli, Paracoccus pantotrophus, Paracoccus rhizosphaerae, Paracoccus saliphilus, Paracoccus sanguinis, Paracoccus sediminis, Paracoccus seriniphilus, Paracoccus solventivorans, Paracoccus sordidisoli, Paracoccus sphaerophysae, Paracoccus stylophorae, Paracoccus sulfuroxidans, Paracoccus thiocyanatus, Paracoccus tibetensis, Paracoccus versutus, Paracoccus yeei, Paracoccus zeaxanthinifaciens and the strains Paracoccus sp..

In a particular embodiment, the bacterium of the genus Paroccocus is a strain Paracoccus sp.

“Bacterium of the genus Fusobacterium” here means an anaerobic filamentous Gram-negative bacterium of the family Fusobacteriaceae. The genus Fusobacterium includes the species Fusobacterium necrophorum, Fusobacterium nucleatum, Fusobacterium polymorphum, and Fusobacterium novum.

In a particular embodiment, the marker used in the context of the invention is chosen from the group constituted of bacteria of the species Micrococcus luteus and bacteria Paracoccus sp.

Marker Metabolite

The marker used in the context of the invention can be chosen from the group constituted of metabolites, in particular produced by the bacteria defined in the section “Marker microbiome” hereinabove, chosen from 3-hydroxy-3-methylglutarate, 3-methylglutarate/2-methylglutarate, 4-guanidinobutanoate, 4-imidazoleacetate, 5-oxoproline, aconitrate, adipate, alanine, alpha-cetoglutarate, arabonate/xylonate, azelate, beta-citrylglutamate, choline, cis-urocanate, citraconate/glutaconate, fructose, fumarate, gamma-glutamylalanine, gamma-glutamylglutamine, gamma-glutamylglycine, gamma-glutamylisoleucine, gamma-glutamylleucine, gamma-glutamylserine, gamma-glutamylthreonine, gamma-glutamyltryptophane, gamma-glutamylvaline, glutarate, glycerate, glycerol-3-phosphate, glycine, isovalerylglycine, kynurenate, lactate, linoleoyl ethanolamide, malate, maleate, malonate, maltose, methionine sulfoxide, methylsuccinate, N-acetylarginine, N-acetylalanine, N-acetylaspartate, N-acetylglycine, N-acetylhistidine, N-acetylphenylalanine, N-acetylthreonine, N-acetylvaline, oleamide, ornithine, palmitamide, pimelate, proline, salicylate, sebacate, serine, suberate, succinate, undecanedioate and S-amino-omega caprolactam.

In a particular embodiment, the metabolite is chosen from kynurenate, 4-imidazoleacetate, maleate, ornithine, 4-guanidinobutanoate, cis-urocanate, malonate, gamma-glutamylleucine, N-acetylarginine and glycerol-3-phosphate.

As is well known to those skilled in the art, one or more of these metabolites can be produced by one or more of the bacteria defined in the section “Marker microbiome” hereinabove.

Indeed, the bacteria of the species Propionibacterium acnes produce as metabolite, among others proline, alanine, glycine, serine, choline, lactate, malate, maleate, alpha-ketoglutarate and succinate.

The bacteria of the family Micrococcaceae produce as metabolite, among others, alanine, serine, fructose and lactate.

The bacteria of the genus Brachybacterium produce as metabolite, among others, proline, glycine, serine, fructose and maltose.

The bacteria of the genus Brevibacterium produce as metabolite, among others, proline, alanine, serine, ornithine, choline, fructose, maltose, fumarate, lactate, malate, glutarate, alpha-ketoglutarate, succinate, malonate, aconitate, adipate, salicylate and sebacate.

The bacteria of the order Burkholderiales produce as metabolite, among others, proline, alanine, glycine, serine, lactate, malate and succinate.

The bacteria of the genus Paracoccus produce as metabolite, among others, proline, alanine, glycine, serine, ornithine, choline, fructose, maltose, fumarate, lactate, malate, alpha-ketoglutarate, succinate, maleate, malonate, aconitate, and N-acetylphenylalanine.

The bacteria of the family Rhodobacteraceae produce as metabolite, among others, alanine, glycine, glutamine, ornithine and succinate.

The bacteria of the genus Fusobacterium produce as metabolite, among others, proline, alanine, glycine, serine, ornithine, choline, fructose, maltose, fumarate, lactate, malate, glutarate, alpha-ketoglutarate and succinate.

The production of these metabolites by these various bacteria is typically described in Palazzi et at (2018) Environ. Int. 121:1341-1354, Thierry et al. (2011) Int. J. Food Microbiol. 149:19-27, Krulwich et at (1976) J. Bacteriol. 127:179-183, Mori et al (1987) Agricultural Biol. Chem. 51:129-138, Levy et al (2017) Current Op. in Microbiol. 25:8-15, Jin et al (2014) Mol. Pharmacol. 85:777-788, Hug et at. (1999) J. Photochem. Photobiol. B 50:66-73 and Wolfe et al. (2016) Curr. Genet. 62:335-341.

Thus in a particular embodiment, aconitate is produced by a bacterium of the genus Parococcus and/or by a bacterium of the genus Brevibacterium.

In a particular embodiment, adipate is produced by a bacterium of the genus Brevibacterium.

In a particular embodiment, alanine is produced by a bacterium of the species Propionibacterium acnes, a bacterium of the family Micrococcaceae, a bacterium of the genus Brevibacterium, a bacterium of the order Burkholderiales, a bacterium of the genus Paracoccus, a bacterium of the family Rhodobacteraceae and/or a bacterium of the genus Fusobacterium.

In a particular embodiment, alpha-ketoglutarate is produced by a bacterium of the species Propionibacterium acnes, a bacterium of the genus Brevibacterium, a bacterium of the genus Paracoccus and/or a bacterium of the genus Fusobacterium.

In a particular embodiment, choline is produced by a bacterium of the species Propionibacterium acnes, a bacterium of the genus Brevibacterium, a bacterium of the genus Paracoccus and/or a bacterium of the genus Fusobacterium.

In a particular embodiment, fructose is produced by a bacterium of the species Propionibacterium acnes, a bacterium of the family Micrococcaceae, a bacterium of the genus Brachybacterium, a bacterium of the genus Brevibacterium, a bacterium of the genus Paracoccus and/or a bacterium of the genus Fusobacterium.

In a particular embodiment, fumarate is produced by a bacterium of the genus Brevibacterium, a bacterium of the genus Paracoccus and/or a bacterium of the genus Fusobacterium.

In a particular embodiment, gamma-glutamylglutamine is produced by a bacterium of the family Rhodobacteraceae.

In a particular embodiment, glutarate is produced by a bacterium of the genus Brevibacterium and/or a bacterium of the genus Fusobacterium.

In a particular embodiment, glycine is produced by a bacterium of the species Propionibacterium acnes, a bacterium of the genus Brachybacterium, a bacterium of the order Burkholderiales, a bacterium of the genus Paracoccus, a bacterium of the family Rhodobacteraceae and/or a bacterium of the genus Fusobacterium.

In a particular embodiment, lactate is produced by a bacterium of the species Propionibacterium acnes, a bacterium of the family Micrococcaceae, a bacterium of the genus Brevibacterium, a bacterium of the order Burkholderiales, a bacterium of the genus Paracoccus, and/or a bacterium of the genus Fusobacterium.

In a particular embodiment, malate is produced by a bacterium of the species Propionibacterium acnes, a bacterium of the genus Brevibacterium, a bacterium of the order Burkholderiales, a bacterium of the genus Paracoccus, and/or a bacterium of the genus Fusobacterium.

In a particular embodiment, maleate is produced by a bacterium of the species Propionibacterium acnes and/or a bacterium of the genus Paracoccus.

In a particular embodiment, malonate is produced by a bacterium of the genus Brevibacterium and/or a bacterium of the genus Paracoccus.

In a particular embodiment, maltose is produced by a bacterium of the genus Brachybacterium, a bacterium of the genus Brevibacterium, a bacterium of the genus Paracoccus, and/or a bacterium of the genus Fusobacterium.

In a particular embodiment, N-acetylphenylalanine is produced by a bacterium of the genus Paracoccus.

In a particular embodiment, ornithine is produced by a bacterium of the genus Brevibacterium, a bacterium of the genus Paracoccus, a bacterium of the family Rhodobacteraceae and/or a bacterium of the genus Fusobacterium.

In a particular embodiment, proline is produced by a bacterium of the species Propionibacterium acnes, a bacterium of the genus Brachybacterium, a bacterium of the genus Brevibacterium, a bacterium of the order Burkholderiales, a bacterium of the genus Paracoccus, and/or a bacterium of the genus Fusobacterium.

In a particular embodiment, salicylate is produced by a bacterium of the genus Brevibacterium.

In a particular embodiment, sebacate is produced by a bacterium of the genus Brevibacterium.

In a particular embodiment, serine is produced by a bacterium of the species Propionibacterium acnes, a bacterium of the family Micrococcaceae, a bacterium of the genus Brachybacterium, a bacterium of the genus Brevibacterium, a bacterium of the order Burkholderiales, a bacterium of the genus Paracoccus, and/or a bacterium of the genus Fusobacterium.

In a particular embodiment, succinate is produced by a bacterium of the species Propionibacterium acnes, a bacterium of the genus Brevibacterium, a bacterium of the order Burkholderiales, a bacterium of the genus Paracoccus, a bacterium of the family Rhodobacteraceae and/or a bacterium of the genus Fusobacterium.

Method of Diagnosing

The method of diagnosing according to the invention is a method for diagnosing esthetic degradations of skin, in particular linked to pollution.

The esthetic degradations of skin are in particular selected from esthetic pigmentary disorders, lack of radiance and heterogeneity of the complexion.

“Esthetic pigmentary disorders” here means a skin aesthetic default resulting from a deficiency or an absence of pigmentation or of an excess or of a heterogeneous distribution of pigmentation. Esthetic pigmentary disorders include in particular an inhomogeneous color of the skin, actinic lentigo, melasma and pigment spots.

In a particular embodiment, the esthetic degradations of skin are selected from pigment spots and an inhomogeneous color of the skin.

The skin is more particularly the skin of the face, in particular the skin of the cheeks and or the forehead, the skin of the neckline, the skin of the neck, the skin of the arms and of the forearms. More preferably, the skin is the skin of the face, in particular the skin of the cheeks and/or of the neck.

In a particular embodiment, the esthetic degradations of skin are linked to pollution, in particular due to pollution.

“Pollution” here means the exposure to particles of matter, in particular to polycyclic aromatic hydrocarbons (PAH).

In a particular embodiment, the pollution is an exposure to particles of matter, in particular to PAH resulting in a level of PAHs and metabolites of PAH in the hair of the subject as follows:
a level of 2-OH-phenanthrene at least 1.8 times higher than a control level,
a level of 3-OH-fluorene at least 1.6 times higher than a control level,
a level of 3-OH-phenanthrene at least 1.7 times higher than a control level,
a level of B-b-fluoranthene at least 1.8 times higher than a control level,
a level of benzo-g-h-i-perylene at least 1.7 times higher than a control level,
a level of fluoranthene at least 2 times higher than a control level, et/ou
a level of pyrene at least 1.6 times higher than a control level,

the control level being typically the level of said compound in the hair of a subject living in a town with little pollution, in particular a town with an air quality index less than 100 for less as than 100 days, in particular less than 85 days, over one year.

The method of diagnosing according to the invention comprises a step (a) of determining, in a skin sample of the subject, the level of at least one marker chosen from the group constituted of (i) bacteria as defined in the section “Marker microbiome” hereinabove and

(ii) metabolites as defined in the section “Marker metabolite” hereinabove.

The level of said at least one marker can be determined by any suitable technique.

In a particular embodiment, said marker is a bacterium as defined in the section “Marker microbiome” hereinabove, and the level of said at least one marker is determined by measuring the level of the corresponding 16S rDNA gene.

Preferably, said marker is a bacterium as defined in the section “Marker microbiome” hereinabove, and the level of said at least one marker is determined by amplification by PCR combined with a sequencing of the region V1-3 of 16S rDNA.

Typically, the genomic bacterial DNA present in the skin sample is extracted then subjected to an amplification by PCR by using primers that target the region V1-3 of bacterial 16S rDNA. The amplicons of 16S rDNA obtained were subjected to a sequencing making it possible to identify the corresponding bacteria. The level of each bacterial 16S rDNA identified is typically measured in parallel by quantitative PCR.

In another particular embodiment, said marker is a metabolite as defined in the section “Marker metabolite” hereinabove, and the level of said at least one marker is determined by liquid chromatography-mass spectrophotometer (LC/MS/MS).

Typically, the skin samples are extracted and subjected to a liquid chromatography-mass spectrometry (LC/MS/MS) analysis. The various metabolites present are typically identified based on the retention index, the correspondence of the mass with the data in a library, and the “forward” and inverse MS/MS scores with respect to the standards, and the level of metabolites is typically measured by quantification of the peaks obtained by LC/MS/MS by using the area under the curve of the primary MS ions.

In a particular embodiment, the method of diagnosing according to the invention further comprises the steps consisting of:
(b) comparing the level of said at least one marker determined in step (a) with a control, and
(c) based on the comparison of step (b), determining if the skin of the subject displays esthetic degradations, in particular linked to pollution.

In a particular embodiment, the control is a reference value.

In a particular embodiment, the reference value is determined by the mean value of the level of said marker in a determined population, for example a population in a defined age-group, and/or having a defined skin type.

In a particular embodiment, the reference value is the mean value of the level of said marker in a population of subjects, in particular subjects as defined hereinbelow, living in a town with little pollution, in particular a town that has an air quality index less than 100 for less than 100 days, in particular less than 85 days, over one year.

In a particular embodiment, the skin of the subject is diagnosed as displaying esthetic degradations, in particular linked to pollution, when:
the level of bacteria of the species Propionibacterium acnes determined in the skin sample of the subject is at least 0.9 times lower than a control level,
the level of bacteria of the family Micrococcaceae determined in the skin sample of the subject is at least 2.2 times higher than a control level,
the level of bacteria of the genus Brachybacterium determined in the skin sample of the subject is at least 1.7 times higher than a control level,
the level of bacteria of the genus Brevibacterium determined in the skin sample of the subject is at least 2.4 times higher than a control level,
the level of bacteria of the order Burkholderiales determined in the skin sample of the subject is at least 1.8 times higher than a control level,
the level of bacteria of the genus Parococcus determined in the skin sample of the subject is at least 1.4 times higher than a control level,
the level of bacteria of the family Rhodobacteraceae determined in the skin sample of the subject is at least 1.4 times higher than a control level, and/or
the level of bacteria of the genus Fusobacterium determined in the skin sample of the subject is at least 0.7 times lower than a control level,

the control level being typically the level of said marker in a skin sample of a subject living in a town with little pollution, in particular a town with an air quality index less than 100 for less than 100 days, in particular less than 85 days, over one year and

the level of the markers being typically determined by measuring the level of the corresponding 16S rDNA gene, in particular by amplification by PCR combined with a sequencing of the region V1-3 of 16S rDNA, typically as described hereinabove.

In a particular embodiment, the skin of the subject is diagnosed as displaying esthetic degradations, in particular linked to pollution, when:
the level of bacteria of the species Micrococcus luteus determined in the skin sample of the subject is at least 1.01 times higher than a control level, and/or
the level of bacteria of the genus Paracoccus determined in the skin sample of the subject is at least 1.4 times higher than a control level,

the control level being typically the level of said marker in a skin sample of a subject living in a town with little pollution, in particular a town with an air quality index less than 100 for less than 100 days, in particular less than 85 days, over one year and

the level of the markers being typically determined by measuring the level of the corresponding 16S rDNA gene, in particular by amplification by PCR combined with a sequencing of the region V1-3 of 16$ rDNA, typically as described hereinabove.

In a particular embodiment, the skin of the subject is diagnosed as displaying esthetic degradations, in particular linked to pollution, when:
the level of 3-hydroxy-3-methylglutarate determined in a skin sample of the subject is at least 1.3 times higher than a control level,
the level of 3-methylglutarate/2-methylglutarate determined in the sample of the skin of the subject is at least 1.3 times higher than a control level,
the level of 4-guanidinobutanoate determined in a skin sample of the subject is at least 1.4 times higher than a control level,
the level of 4-imidazoleacetate determined in a skin sample of the subject is at least 1.4 times higher than a control level,
the level of 5-oxoproline determined in a skin sample of the subject is at least 1.3 times higher than a control level,
the level of aconitrate determined in a skin sample of the subject is at least 1.4 times higher than a control level,
the level of adipate determined in a skin sample of the subject is at least 1.2 times higher than a control level,
the level of alanine determined in a skin sample of the subject is at least 1.3 times higher than a control level,
the level of alpha-ketoglutarate determined in a skin sample of the subject is at least 1.1 times higher than a control level,
the level of arabonate/xylonate determined in a skin sample of the subject is at least 1.4 times higher than a control level,
the level of azelate determined in a skin sample of the subject is at least 1.2 times higher than a control level,
the level of beta-citrylglutamate determined in a skin sample of the subject is at least 1.3 times higher than a control level,
the level of choline determined in a skin sample of the subject is at least 1.2 times higher than a control level,
the level of cis-urocanate determined in a skin sample of the subject is at least 1.5 times higher than a control level,
the level of citraconate/glutaconate determined in a skin sample of the subject is at least 1.3 times higher than a control level,
the level of fructose determined in a skin sample of the subject is at least 1.1 times higher than a control level,
the level of fumarate determined in a skin sample of the subject is at least 1.2 times higher than a control level,
the level of gamma-glutamylalanine determined in a skin sample of the subject is at least 1.3 times greater than a control level,
the level of gamma-glutamylglutamine determined in a skin sample of the subject is at least 1.2 times higher than a control level,
the level of gamma-glutamylglycine determined in a skin sample of the subject is at least 1.4 times higher than a control level,
the level of gamma-glutamylisoleucine determined in a skin sample of the subject is at least 1.3 times higher than a control level,
the level of gamma-glutamylleucine determined in a skin sample of the subject is at least 1.4 times higher than a control level,
the level of gamma-glutamylserine determined in a skin sample of the subject is at least 1.4 times higher than a control level,
the level of gamma-glutamylthreonine determined in a skin sample of the subject is at least 1.3 times higher than a control level,
the level of gamma-glutamyltryptophane determined in a skin sample of the subject is at least 1.4 times higher than a control level,
the level of gamma-glutamylvaline determined in a skin sample of the subject is at least 1.4 times higher than a control level,
the level of glutarate determined in a skin sample of the subject is at least 1.1 times higher than a control level,
the level of glycerate determined in a skin sample of the subject is at least 1.1 times higher than a control level,
the level of glycerol-3-phosphate determined in a skin sample of the subject is at least 1.6 times higher than a control level,
the level of glycine determined in a skin sample of the subject is at least 1.3 times higher than a control level,
the level of isovalerylglycine determined in a skin sample of the subject is at least 1.3 times higher than a control level,
the level of kynurenate determined in a skin sample of the subject is at least 2.3 times higher than a control level,
the level of lactate determined in a skin sample of the subject is at least 1.2 times higher than a control level,
the level of linoleoyl ethanolamide determined in a skin sample of the subject is at least 1.3 times higher than a control level,
the level of malate determined in a skin sample of the subject is at least 1.2 times higher than a control level,
the level of maleate determined in a skin sample of the subject is at least 1.5 times higher than a control level,
the level of malonate determined in a skin sample of the subject is at least 1.4 times higher than a control level,
the level of maltose determined in a skin sample of the subject is at least 1.6 times higher than a control level,
the level of methionine sulfoxide determined in a skin sample of the subject is at least 1.2 times higher than a control level,
the level of methylsuccinate determined in a skin sample of the subject is at least 1.3 times higher than a control level,
the level of N-acetylarginine determined in the skin sample of the subject is at least 1.6 times higher than a control level,
the level of N-acetylalanine determined in a skin sample of the subject is at least 1.2 times higher than a control level,
the level of N-acetylaspartate determined in a skin sample of the subject is at least 1.4 times higher than a control level,
the level of N-acetylglycine determined in a skin sample of the subject is at least 1.3 times higher than a control level,
the level of N-acetylhistidine determined in a skin sample of the subject is at least 1.2 times higher than a control level,
the level of N-acetylphenylalanine determined in a skin sample of the subject is at least 1.3 times higher than a control level,
the level of N-acetylthreonine determined in a skin sample of the subject is at least 1.1 times higher than a control level,
the level of N-acetylvaline determined in a skin sample of the subject is at least 1.2 times higher than a control level,
the level of oleamide determined in a skin sample of the subject is at least 1.3 times higher than a control level,
the level of ornithine determined in a skin sample of the subject is at least 1.3 times higher than a control level,
the level of palmitamide determined in a skin sample of the subject is at least 1.4 times higher than a control level,
the level of pimelate determined in a skin sample of the subject is at least 1.2 times higher than a control level,
the level of proline determined in a skin sample of the subject is at least 1.3 times higher than a control level,
the level of salicylate determined in a skin sample of the subject is at least 1.5 times higher than a control level,
the level of sebacate determined in a skin sample of the subject is at least 1.2 times higher than a control level,
the level of serine determined in a skin sample of the subject is at least 1.3 times higher than a control level,
the level of suberate determined in a skin sample of the subject is at least 1.2 times higher than a control level,
the level of succinate determined in a skin sample of the subject is at least 1.2 times higher than a control level,
the level of undecanedioate determined in a skin sample of the subject is at least 1.3 times higher than a control level, and/or
the level of S-amino-omega caprolactam determined in a skin sample of the subject is at least 2.3 times higher than a control level;

the control level being typically the level of said marker in a skin sample of a subject living in a town with little pollution, in particular a town with an air quality index less than 100 for less than 100 days, in particular less than 85 days, over one year and the level of the markers being typically determined by liquid chromatography-mass spectrometry (LC/MS/MS), typically as described hereinabove.

In a particular embodiment, the skin of the subject is diagnosed as displaying esthetic degradations, in particular linked to pollution, when:
the level of kynurenate determined in a skin sample of the subject is at least 2.3 times higher than a control level,
the level of 4-imidazoleacetate determined in a skin sample of the subject is at least 1.4 times higher than a control level,
the level of maleate determined in a skin sample of the subject is at least 1.5 times higher than a control level,
the level of ornithine determined in a skin sample of the subject is at least 1.3 times higher than a control level,
the level of 4-guanidinobutanoate determined in a skin sample of the subject is at least 1.4 times higher than a control level,
the level of cis-urocanate determined in a skin sample of the subject is at least 1.5 times higher than a control level,
the level of malonate determined in a skin sample of the subject is at least 1.4 times higher than a control level,
the level of gamma-glutamylleucine determined in a skin sample of the subject is at least 1.4 times higher than a control level, the level of N-acetylarginine determined in a skin sample of the subject is at least 1.6 times higher than a control level, and/or
the level of glycerol-3-phosphate determined in a skin sample of the subject is at least 1.6 times higher than a control level,

the control level being typically the level of said marker in a skin sample of a subject living in a town with little pollution, in particular a town with an air quality index less than 100 for less than 100 days, in particular less than 85 days, over one year and

the level of the markers being typically determined by liquid chromatography-mass spectrometry (LC/MS/MS), typically as described hereinabove.

According to a preferred embodiment, the skin sample of the subject used in the method of diagnosing according to the invention is a sample taken, preferably non-invasively, on the subject's skin, preferentially on the subject's face, in particularly on the subject's cheek and/or forehead. Preferably, the skin sample is from the stratum corneum.

The stratum corneum is the outermost layer of the epidermis, and comprises the skin surface. It is mainly made up of dead cells.

According to an embodiment, the method of diagnosing according to the invention comprises a step of taking the skin sample from the subject. This step is preferably performed non-invasively, and in particular does not require local anesthetic. According to a preferred embodiment, the step of taking the sample is performed by rubbing the skin surface or using an adhesive surface such as a D-squame® disc.

In a particular embodiment, the skin sample is taken using a D-squame@ disc.

“Subject” here means a human being, preferably aged 25 to 45 years. Preferably, the subject is female. Preferably, the subject is of the Asiatic type.

The present invention also relates to a method of cosmetic treatment of a skin displaying esthetic degradations of skin, in particular linked to pollution, in a subject, said method comprising the following steps:
A) diagnosing the subject as displaying esthetic degradations of skin, in particular linked to pollution, by implementing the method of diagnosing according to the invention
B) if the subject is diagnosed as displaying esthetic degradations of skin, in particular linked to pollution, treating the skin of said subject with a cosmetic composition allowing for the reduction and/or the slowing down of the esthetic degradations of skin.

Method for Evaluating the Cutaneous Exposure to Pollution

The present invention also relates to a method for evaluating the cutaneous exposure of a subject to pollution, comprising a step (a) of determining, in a skin sample of the subject, the level of at least one marker chosen from the group constituted of (i) bacteria as defined in the section “Marker microbiome” hereinabove and (ii) metabolites as defined in the section “Marker metabolite” hereinabove.

The pollution is as defined in the section “Method of diagnosing” hereinabove.

The step (a) of determining is preferably implemented as described hereinabove for the method of diagnosing.

The skin sample is as defined in the section “Method of diagnosing” hereinabove.

The subject is as defined in the section “Method of diagnosing” hereinabove.

In a particular embodiment, the method of evaluating according to the invention further comprises the steps consisting of:
(b) comparing the level of said at least one marker determined in step (a) with a control, and
(c) based on the comparison of step (b), determining if the subject has been subjected to a cutaneous exposure to pollution.

In a particular embodiment, the control is a reference value.

In a particular embodiment, the reference value is determined by the mean value of the level of said marker in a determined population, for example a population in a defined age-group, and/or having a defined skin type.

In a particular embodiment, the reference value is the mean value of the level of said marker in a population of subjects, in particular subjects as defined hereinbelow, living in a town with little pollution, in particular a town that has an air quality index less than 100 for less than 100 days, in particular less than 85 days, over one year.

In a particular embodiment, the subject is determined as having been subjected to a cutaneous exposure to pollution, when:
the level of bacteria of the species Propionibacterium acnes determined in the skin sample of the subject is at least 0.9 times lower than a control level,
the level of bacteria of the family Micrococcaceae determined in the skin sample of the subject is at least 2.2 times higher than a control level,
the level of bacteria of the genus Brachybacterium determined in the skin sample of the subject is at least 1.7 times higher than a control level,
the level of bacteria of the genus Brevibacterium determined in the skin sample of the subject is at least 2.4 times higher than a control level,
the level of bacteria of the order Burkholderiales determined in the skin sample of the subject is at least 1.8 times higher than a control level,
the level of bacteria of the genus Parococcus determined in the skin sample of the subject is at least 1.4 times higher than a control level,
the level of bacteria of the family Rhodobacteraceae determined in the skin sample of the subject is at least 1.4 times higher than a control level, and/or
the level of bacteria of the genus Fusobacterium determined in the skin sample of the subject is at least 0.7 times higher than a control level,

the control level being typically the level of said marker in a skin sample of a subject living in a town with little pollution, in particular a town with an air quality index less than 100 for less than 100 days, in particular less than 85 days, over one year and

the level of the markers being typically determined by measuring the level of the corresponding 16S rDNA gene, in particular by amplification by PCR combined with a sequencing of the region V1-3 of 16S rDNA, typically as described hereinabove.

In a particular embodiment, the subject is determined as having been subjected to a cutaneous exposure to pollution, when:
the level of bacteria of the species Micrococcus luteus determined in the skin sample of the subject is at least 1.01 times higher than a control level, and/or
the level of bacteria of the genus Paracoccus determined in the skin sample of the subject is at least 1.4 times higher than a control level,

the control level being typically the level of said marker in a skin sample of a subject living in a town with little pollution, in particular a town with an air quality index less than 100 for less than 100 days, in particular less than 85 days, over one year and

the level of the markers being typically determined by measuring the level of the corresponding 16S rDNA gene, in particular by amplification by PCR combined with a sequencing of the region V1-3 of 16S rDNA, typically as described hereinabove.

In a particular embodiment, the subject is determined as having been subjected to a cutaneous exposure to pollution, when:
the level of 3-hydroxy-3-methylglutarate determined in a skin sample of the subject is at least 1.3 times higher than a control level,
the level of 3-methylglutarate/2-methylglutarate determined in the sample of the skin of the subject is at least 1.3 times higher than a control level,
the level of 4-guanidinobutanoate determined in a skin sample of the subject is at least 1.4 times higher than a control level,
the level of 4-imidazoleacetate determined in a skin sample of the subject is at least 1.4 times higher than a control level,
the level of 5-oxoproline determined in a skin sample of the subject is at least 1.3 times higher than a control level,
the level of aconitrate determined in a skin sample of the subject is at least 1.4 times higher than a control level,
the level of adipate determined in a skin sample of the subject is at least 1.2 times higher than a control level,
the level of alanine determined in a skin sample of the subject is at least 1.3 times higher than a control level,
the level of alpha-ketoglutarate determined in a skin sample of the subject is at least 1.1 times higher than a control level,
the level of arabonate/xylonate determined in a skin sample of the subject is at least 1.4 times higher than a control level,
the level of azelate determined in a skin sample of the subject is at least 1.2 times higher than a control level,
the level of beta-citrylglutamate determined in a skin sample of the subject is at least 1.3 times higher than a control level,
the level of choline determined in a skin sample of the subject is at least 1.2 times higher than a control level,
the level of cis-urocanate determined in a skin sample of the subject is at least 1.5 times higher than a control level,
the level of citraconate/glutaconate determined in a skin sample of the subject is at least 1.3 times higher than a control level,
the level of fructose determined in a skin sample of the subject is at least 1.1 times higher than a control level,
the level of fumarate determined in a skin sample of the subject is at least 1.2 times higher than a control level,
the level of gamma-glutamylalanine determined in a skin sample of the subject is at least 1.3 times higher than a control level,
the level of gamma-glutamylglutamine determined in a skin sample of the subject is at least 1.2 times higher than a control level,
the level of gamma-glutamylglycine determined in a skin sample of the subject is at least 1.4 times higher than a control level,
the level of gamma-glutamylisoleucine determined in a skin sample of the subject is at least 1.3 times higher than a control level,
the level of gamma-glutamylleucine determined in a skin sample of the subject is at least 1.4 times higher than a control level,
the level of gamma-glutamylserine determined in a skin sample of the subject is at least 1.4 times higher than a control level,
the level of gamma-glutamylthreonine determined in a skin sample of the subject is at least 1.3 times higher than a control level,
the level of gamma-glutamyltryptophane determined in a skin sample of the subject is at least 1.4 times higher than a control level,
the level of gamma-glutamylvaline determined in a skin sample of the subject is at least 1.4 times higher than a control level,
the level of glutarate determined in a skin sample of the subject is at least 1.1 times higher than a control level,
the level of glycerate determined in a skin sample of the subject is at least 1.1 times higher than a control level,
the level of glycerol-3-phosphate determined in a skin sample of the subject is at least 1.6 times higher than a control level,
the level of glycine determined in a skin sample of the subject is at least 1.3 times higher than a control level,
the level of isovalerylglycine determined in a skin sample of the subject is at least 1.3 times higher than a control level,
the level of kynurenate determined in a skin sample of the subject is at least 2.3 times higher than a control level,
the level of lactate determined in a skin sample of the subject is at least 1.2 times higher than a control level,
the level of linoleoyl ethanolamide determined in a skin sample of the subject is at least 1.3 times higher than a control level,
the level of malate determined in a skin sample of the subject is at least 1.2 times higher than a control level,
the level of maleate determined in a skin sample of the subject is at least 1.5 times higher than a control level,
the level of malonate determined in a skin sample of the subject is at least 1.4 times higher than a control level,
the level of maltose determined in a skin sample of the subject is at least 1.6 times higher than a control level,
the level of methionine sulfoxide determined in a skin sample of the subject is at least 1.2 times higher than a control level,
the level of methylsuccinate determined in a skin sample of the subject is at least 1.3 times higher than a control level,
the level of N-acetylarginine determined in a skin sample of the subject is at least 1.6 times higher than a control level,
the level of N-acetylalanine determined in a skin sample of the subject is at least 1.2 times higher than a control level,
the level of N-acetylaspartate determined in a skin sample of the subject is at least 1.4 times higher than a control level,
the level of N-acetylglycine determined in a skin sample of the subject is at least 1.3 times higher than a control level,
the level of N-acetylhistidine determined in a skin sample of the subject is at least 1.2 times higher than a control level,
the level of N-acetylphenylalanine determined in a skin sample of the subject is at least 1.3 times higher than a control level,
the level of N-acetylthreonine determined in a skin sample of the subject is at least 1.1 times higher than a control level,
the level of N-acetylvaline determined in a skin sample of the subject is at least 1.2 times higher than a control level,
the level of oleamide determined in a skin sample of the subject is at least 1.3 times higher than a control level,
the level of ornithine determined in a skin sample of the subject is at least 1.3 times higher than a control level,
the level of palmitamide determined in a skin sample of the subject is at least 1.4 times higher than a control level,
the level of pimelate determined in a skin sample of the subject is at least 1.2 times higher than a control level,
the level of proline determined in a skin sample of the subject is at least 1.3 times higher than a control level,
the level of salicylate determined in a skin sample of the subject is at least 1.5 times higher than a control level,
the level of sebacate determined in a skin sample of the subject is at least 1.2 times higher than a control level,
the level of serine determined in a skin sample of the subject is at least 1.3 times higher than a control level,
the level of suberate determined in a skin sample of the subject is at least 1.2 times higher than a control level,
the level of succinate determined in a skin sample of the subject is at least 1.2 times higher than a control level,
the level of undecanedioate determined in a skin sample of the subject is at least 1.3 times higher than a control level, and/or
the level of S-amino-omega caprolactam determined in a skin sample of the subject is at least 2.3 times higher than a control level;

the control level being typically the level of said marker in a skin sample of a subject living in a town with little pollution, in particular a town with an air quality index less than 100 for less than 100 days, in particular less than 85 days, over one year and

the level of the markers being typically determined by liquid chromatography-mass spectrometry (LC/MS/MS), typically as described hereinabove.

In a particular embodiment, the subject is determined as having been subjected to a cutaneous exposure to pollution, when:
the level of kynurenate determined in a skin sample of the subject is at least 2.3 times higher than a control level,
the level of 4-imidazoleacetate determined in a skin sample of the subject is at least 1.4 times higher than a control level,
the level of maleate determined in a skin sample of the subject is at least 1.5 times higher than a control level,
the level of ornithine determined in a skin sample of the subject is at least 1.3 times higher than a control level,
the level of 4-guanidinobutanoate determined in a skin sample of the subject is at least 1.4 times higher than a control level,
the level of cis-urocanate determined in a skin sample of the subject is at least 1.5 times higher than a control level,
the level of malonate determined in a skin sample of the subject is at least 1.4 times higher than a control level,
the level of gamma-glutamylleucine determined in a skin sample of the subject is at least 1.4 times higher than a control level,
the level of N-acetylarginine determined in a skin sample of the subject is at least 1.6 times higher than a control level, and/or
the level of glycerol-3-phosphate determined in a skin sample of the subject is at least 1.6 times higher than a control level,

the control level being typically the level of said marker in a skin sample of a subject living in a town with little pollution, in particular a town with an air quality index less than 100 for less than 100 days, in particular less than 85 days, over one year and the level of the markers being typically determined by liquid chromatography-mass spectrometry (LC/MS/MS), typically as described hereinabove.

The present invention will be described in more detail by the figures and examples hereinbelow.

### DESCRIPTION OF THE SEQUENCES

| OTU | Taxonomy | SEQ ID | Sequence |
|---|---|---|---|
| B1 | P. acnes | 1 | AGAGTTTGATCCTGGCTCAGGACGAACGCTGGCGGCGTGCTT |
| | | | AACACATGCAAGTCGAACGGAAAGGCCCTGCTTTTGTGGGGT |
| | | | GCTOGAGTGGCGAACGGGTGAGTAACACGTGAGTAACCTGCC |
| | | | CTTGACTTTGGGATAACTTCAGGAAACTGGGGCTAATACCGGA |
| | | | TAGGAGCTCCTGCTGCATGGTGGGGGTTGGAAAGTTTCGGCG |
| | | | GTTGGGGATGGACTCGCGGCTTATCAGCTTGTTGGTGGGGTA |
| | | | GTGGCTTACCAAGGCTTTGACGGGTAGCCGGCCTGAGAGGGT |
| | | | GACCGGCCACATTGGGACTGAGATACGGCCCAGACTCCTACG |
| | | | GGAGGCAGCAGTGGGGAATATTGCACAATGGGCGGAAGCCT |
| | | | GATGCAGCAACGCCGCGTGCGGGATGACGGCCTTCGGGTTG |
| | | | TAAACCGCTTTCGCCTGTGACGAAGCGTGAG |
| B260 | Micrococcaceae | 2 | AGAGTTTGATCCTGGCTCAGGATGAACGCTGGCGGCGTGCTT |
| | (family) | | AACACATGCAAGTCGAACGATGAAGCCCAGCTTGCTGGGTGG |
| | | | ATTAGTGGCGAACGGGTGAGTAACACGTGAGTAACCTGCCCT |
| | | | TGACTCTGGGATAAGCCTGGGAAACTGGGTCTAATACTGGATA |
| | | | TGACTTCTCACCGCATGGTGGGGGGTGGAAAGATTTATTGGTC |
| | | | TTGGATGGACTCGCGGCCTATCAGCTTGTTGGTGAGGTAATG |
| | | | GCTCACCAAGGCGACGACGGGTAGCCGGCCTGAGAGGGTGA |
| | | | CCGGCCACACTGGGACTGAGACACGGCCCAGACTCCTACGG |
| | | | GAGGCAGCAGTGGGGAATATTGCACAATGGGCGCAAGCCTGA |
| | | | TGCAGCGACGCCGCGTGAGGGATGACGGCCTTCGGGTTGTAA |
| | | | ACCTCTTTCAGTAGGGAAGAAGCCTTCGGG |
| B32 | Brachybacterium | 3 | AGAGTTTGATCCTGGCTCAGGACGAACGCTGGCGGCGTGCTT |
| | (genus) | | AACACATGCAAGTCGAACGATGACGACAGGGCTTGCCTTGTCT |
| | | | GATTAGTGGCGAACGGGTGAGTAACACGTGAGTAACCTGCCC |
| | | | CCCTCTTCGGGATAACCTCGGGAAATCGGGGCTAATACCGGA |
| | | | TATGACTTCGCACCGCATGGTGTGTTGTGGAAAGTTTTTCGGT |
| | | | GGGGGATGGACTCGCGGCCTATCAGTTTGTTGGTGAGGTAAT |
| | | | GGCTCACCAAGGCGATGACGGGTAGCCGGCCTGAGAGGGCG |
| | | | ACCGGCCACACTGGGACTGAGACACGGCCCAGACTCCTACGG |
| | | | GAGGCAGCAGTGGGGAATATTGCACAATGGGCGAAAGCCTGA |
| | | | TGCAGCGACGCCGCGTGGGGGATGACGGCCTTCGGGTTGTA |
| | | | AACCTCTTTCAGTAGGGAAGAAGCGAAAGT |
| B37 | Brevibacterium | 4 | AGAGTTTGATCCTGGCTCAGGACGAACGCTGGCGGCGTGCTT |
| | (genus) | | AACACATGCAAGTCGAACGCTGAAGCCTGCAGCTTGCTGTGG |
| | | | GTGGATGAGTGGCGAACGGGTGAGTAACACGTGAGTAACCTG |
| | | | CCCCTGACTCTGGGATAAGCCCGGGAAACTGGGTCTAATACC |
| | | | GGATATGACCATTCTCCGCATGGAGTGGTGGTGGAAAGTTTTT |
| | | | CGGTTGGGGATGGGCTCGCGGCCTATGAGCTTGTTGGTGGG |
| | | | GTGATGGCCTACCAAGGCGACGACGGGTAGCCGGCCTGAGA |
| | | | GGGCGACCGGCCACACTGGGACTGAGACACGGCCCAGACTC |
| | | | CTACGGGAGGCAGCAGTGGGGAATATTGCACAATGGGGGAAA |
| | | | CCCTGATGCAGCGACGCCGCGTGGGGGATGACGGCCTTCGG |
| | | | GTTGTAAACCTCTTTCAGTAGGGAAGAAGCCTT |
| B41 | Burkholderiales | 5 | AGAGTTTGATCCTGGCTCAGATTGAACGCTGGCGGCATGCGTT |
| | (order) | | ACACATGCAAGTCGAACGGCAGCATGATCTAGCTTGCTAGATT |
| | | | GATGGCGAGTGGCGAACGGGTGAGTAATACATCGGAACGTGC |
| | | | CCTGTAGTGGGGGATAACTAGTCGAAAGATTAGCTAATACCGC |
| | | | ATACGACCTGAGGGTGAAAGTGGGGGACCGCAAGGCCTCATG |
| | | | CTATAGGAGCGGCCGATGTCTGATTAGCTAGTTGGTGGGGTA |
| | | | AAGGCCCACCAAGGCGACGATCAGTAGCTGGTCTGAGAGGAC |
| | | | GATCAGCCACACTGGGACTGAGACACGGCCCAGACTCCTACG |
| | | | GGAGGCAGCAGTGGGGAATTTTGGACAATGGGGGCAACCCTG |
| | | | ATCCAGCAATGCCGCGTGTGTGAAGAAGGCCTTCGGGTTGTA |
| | | | AAGCACTTTTGTCCGGAAAGAAATCGC |
| B52 | Paracoccus | 6 | AGAGTTTGATCCTGGCTCAGAACGAACGCTGGCGGCAGGCCT |
| | (genus) | | AACACATGCAAGTCGAGCGCGCCCTTCGGGGTGAGCGGCGG |
| | | | ACGGGTGAGTAACGCGTGGGAACGTGCCCTTCTCTGCGGAAT |
| | | | AGCCTCGGGAAACTGAGAGTAATACCGCATACGCCCTTTGGG |
| | | | GGAAAGATTTATCGGAGAAGGATCGGCCCGCGTTGGATTAGG |
| | | | TAGTTGGTGGGGTAATGGCCCACCAAGCCGACGATCCATAGC |
| | | | TGGTTTGAGAGGATGATCAGCCACACTGGGACTGAGACACGG |
| | | | CCCAGACTCCTACGGGAGGCAGCAGTGGGGAATCTTAGACAA |
| | | | TGGGGGCAACCCTGATCTAGCCATGCCGCGTGAGTGATGAAG |
| | | | GCCTTAGGGTTGTAAAGCTCTTTCAGCTGGGAAGATAATGACG |
| | | | GTACCAGCAGAAGAAGCCCCGGCTAACTCC |
| B81 | Rhodobacteraceae | 7 | AGAGTTTGATCCTGGCTCAGAACGAACGCTGGCGGCAGGCTT |
| | (family) | | AACACATGCAAGTCGAGCGAGATCTTCGGGTCTAGCGGCGGA |
| | | | CGGGTGAGTAACGCGTGGGAACGTGCCCTTTGCTACGGAATA |
| | | | GTCCTGGGAAACTGGGGGTAATACCGTATACGCCCTTTTGGG |
| | | | GAAAGATTTATCGGCGAAGGATCGGCCCGCGTTGGATTAGGT |
| | | | AGTTGGTGGGGTAATGGCCTACCAAGCCGACGATCCATAGCT |
| | | | GGTTTGAGAGGATGATCAGCCACACTGGGACTGAGACACGGC |
| | | | CCAGACTCCTACGGGAGGCAGCAGTGGGGAATCTTAGACAAT |
| | | | GGGGGCAACCCTGATCTAGCCATGCCGCGTGAGTGATGAAGG |
| | | | GCTTAGGGTTGTAAAGCTGTTTGAGCTGGGAAGATAATGACGG |
| | | | TACCAGCAGAAGAAGCCCCGGCTAACTCC |
| 885 | Fusobacterium | 8 | AGAGTTTGATCCTGGCTCAGGATGAACGCTGACAGAATGCTTA |
| | (genus) | | ACACATGCAAGTCTACTTGAACTTCGGTTTGGGTGGCGGACG |
| | | | GGTGAGTAACGCGTAAAGAACTTGCCTCACAGTTAGGGACAA |
| | | | CATTTGGAAACGAATGCTAATACCTGATATTATGAT7TTAGGGC |
| | | | ATCCTAAGATTATGAAAGCTATATGCGCTGTGAGAGAGCTTTG |
| | | | CGTCCCATTAGCTAGTTGGAGAGGTAACGGCTCACCAAGGCG |
| | | | ATGATGGGTAGCCGGCCTGAGAGGGTGAACGGCCACAAGGG |
| | | | GACTGAGACACGGCCCTTACTCCTACGGGAGGCAGCAGTGGG |
| | | | GAATATTGGACAATGGACCAAAAGTCTGATCCAGCAATTCTGT |
| | | | GTGCACGATGACGTTTTTCGGAATGTAAAGTGCTTTCAGTTGG |
| | | | GAAGAAAAAAATGACGGTACCAACA |
| B11 | Micrococcus | 9 | AGAGTTTGATCCTGGCTCAGGATGAACGCTGGCGGCGTGCTT |
| | luteus (species) | | AACACATGCAAGTCGAACGATGAAGCCCAGCTTGCTGG |
| | | | GTGGATTAGTGGCGAACGGGTGAGTAACACGTGAGTAACCTG |
| | | | CCCTTAACTCTGGGATAAGCCTGGGAAACTGGGTCTAA |
| | | | TACCGGATAGGAGCGTCCACCGCATGGTGGGTGTTGGAAAGA |
| | | | TTTATCGGTTTTGGATGGACTCGCGGCCTATCAGCTTG |
| | | | TTGGTGAGGTAATGGCTCACCAAGGCGACGACGGGTAGCCGG |
| | | | CCTGAGAGGGTGACCGGCCACACTGGGACTGAGACACG |
| | | | GCCCAGACTCCTACGGGAGGCAGCAGTGGGGAATATTGCACA |
| | | | ATGGGCGCAAGCCTGATGCAGCGACGCCGCGTGAGGGA |
| | | | TGACGGCCTTCGGGTTGTAAACCTCTTTCAGTAGGGAAGAAGC |
| | | | GAAAGTG |

### BRIEF DESCRIPTION OF THE FIGURES

FIG. 1: Appearance and severity of the extended maculae on the cheek and the forehead of women in the groups GP1 and GP2 defined in the examples (mean scores).

FIG. 2: Severity of the extended maculae over a clinical score from 1 to 4 (4 being the most severe) within each group.

### EXAMPLE

The example hereinbelow shows the identification of signatures comprising 60 metabolites and 8 microbes which are significantly modulated in skin samples of individuals exposed to chronic pollution (based on the detection of high levels of pollutants in the samples of their hair).

Methods and Results

identification of the “polluted” vs. “non-polluted” groups

Two groups of individuals, called Group 1 (GP1) and Group 2 (GP2) in the text hereinbelow, were derived from an integrative unsupervised multivariate analysis of blocks of PAH data (polycyclic aromatic hydrocarbons), metabolites and bacterial microbiome.

Skin samples (D-Squame®) from 42 women (GP1) exposed to a high level of pollution were compared with those of 45 women (GP2) exposed to a relatively low level of pollution. The women of the two groups were in good health, and aged 25-45 years. 30/42 women in Group 1 (GP1) lived in a polluted city in China for at least 15 years. 42/45 women in Group 2 (GP2) lived in a less polluted city for at least 15 years.

The polluted and non-polluted cities were selected based on a significantly discriminating Air Quality Index (AQI) over a period of one year.

Here, pollution is defined as exposure to particles of matter and was established in these women by analyzing the PAHs and metabolites of PAHs in hair samples (Palazzi et al. (2018) Environment international 121:1341-1354).

The PAHs and metabolites of PAHs were quantified in pg/ml and compared between the two groups. A V-test analysis was conducted and the Fold Change (F.C.) was calculated. The statistical analysis is described in a separate section.

TABLE
F.C. PAH and PAH metabolite, GP1 versus GP2

| | PAH and PAH | Log2 F.C. | F.C. | |
|---|---|---|---|---|
| No. | metabolites | (GP1 vs GP2) | (GP1 vs GP2) | p value |
| 1 | In_2OHPhenanth | 0.872588218 | 1.8309447 | 0.000738291 |
| 2 | In_3OHFluorene | 0.730025913 | 1.658668884 | 0.001879259 |
| 3 | In_3OHPhenanth | 0.782246456 | 1.719806742 | 0.002666112 |
| 4 | In_Bbfluora | 0.900554433 | 1.866783257 | 6.19299 × 10⁻⁷ |
| 5 | In_Benzoghipery | 0.812510839 | 1.756265356 | 8.39888 × 10⁻⁸ |
| 6 | In_fluoranthene | 1.061596344 | 2.087239778 | 2.70474 × 10⁻¹⁴ |
| 7 | In_pyrene | 0.757862725 | 1.690983667 | 2.03191 × 10⁻¹¹ |

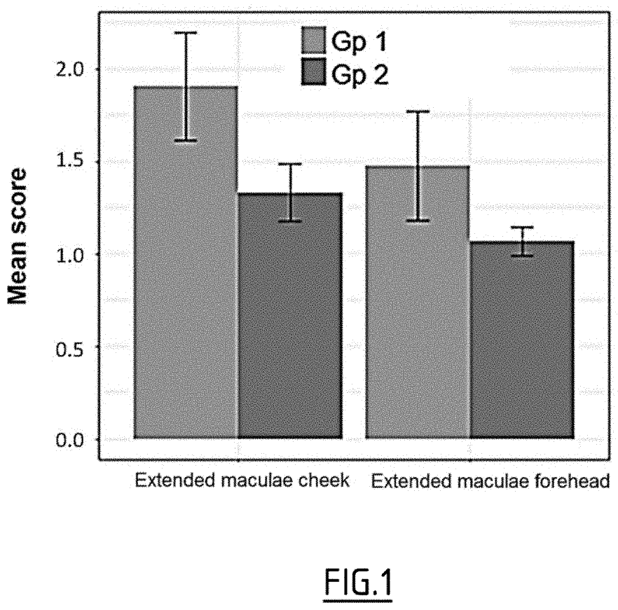

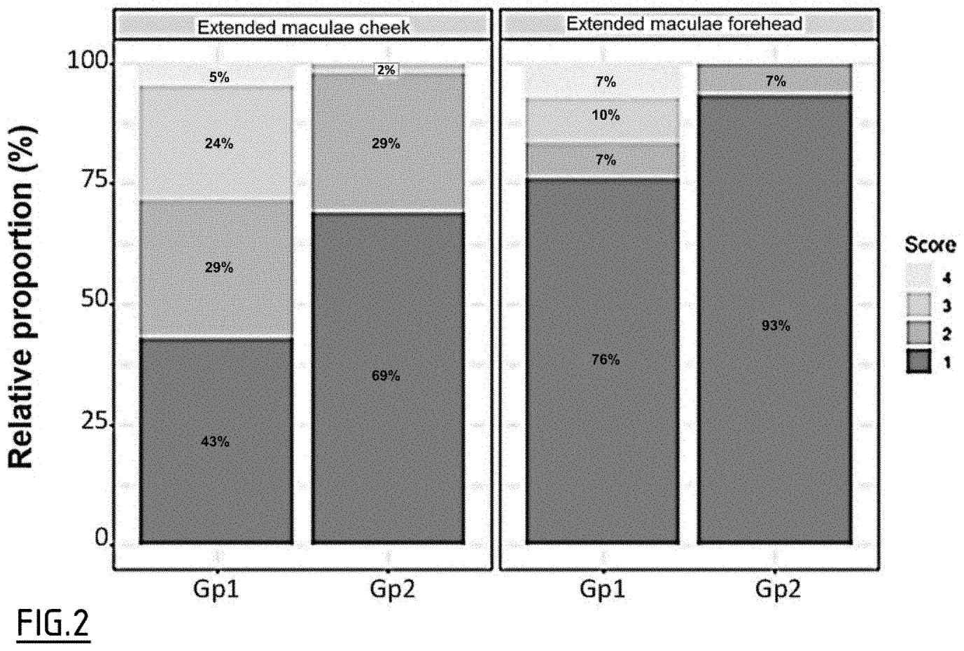

The women in GP1 have the clinical sign “Extended maculae or inhomogeneous skin color” on the cheek and on the forehead in a higher way, with respect to the women in GP2 (see FIG. 1). The severity of this clinical sign, evaluated over a score ranging from 1 to 4, is j higher in GP1 with respect to GP2 (see FIG. 2).

Analysis of the Metabolites

An analysis of the metabolites was conducted on D-Squame® samples of GP1 and GP2. All the samples were analyzed using a non-targeted approach.

In sum, the samples were extracted and divided into equal parts for an analysis on liquid chromatography-mass spectrometry (LC/MS/MS) and polar liquid chromatography platforms. A suitable software was used to have the ions correspond to a customized library of standards for the identification of metabolites and for the quantification of metabolites by integration of the area of the peak.

The biochemical identifications are therefore based on 3 criteria: the retention index within a narrow retention window of the identification proposed, a precise correspondence of the mass with the library ±10 ppm, and the “forward” and inverse MS/MS scores between the experimental data and the authentic standards. The MS/MS scores are based on a comparison of the ions present in the experimental spectrum and the ions present in the spectrum of the library. Although there may be similarities between the molecules based on one of these factors, using three data points makes it possible to distinguish and to differentiate the biochemical molecules.

More than 3300 commercially-available purified standard compounds were acquired and recorded in the LIMS for analysis on all the platforms in order to determine their analytical characteristics. Additional entries of mass spectra were created for the structurally unnamed biochemical molecules, which were identified through their recurring nature (both chromatographically and mass spectrum). These compounds have the potential of being identified by future acquisition of a corresponding purified standard or by conventional structural analysis. The peaks were quantified by using the area under the curve of the primary MS ions.

Conventional T-tests were used to determine the statistical significance.

A variety of treatment procedures was implemented in order to ensure that a high-quality dataset was made available for the statistical analyses and data interpretation. The quality control and treatment processes were designed to ensure precise and coherent identification of the true chemical entities, and to eliminate those representing artifacts of the system, incorrect assignments and background noise.

TABLE
F.C. Metabolite, GP1 versus GP2

| | | Log2 F.C. | F.C. | |
|---|---|---|---|---|
| No. | Metabolite | (GP1 vs GP2) | (GP1 vs GP2) | p value |
| 1 | 3-hydroxy-3-methylglutarate | 0.412283837 | 1.330790837 | 0.000141773 |
| 2 | 3-methylglutarate/2-methylglutarate | 0.419605244 | 1.337561516 | 2.01884E−05 |
| 3 | 4-guanidinobutanoate | 0.493307598 | 1.407668473 | 0.013184653 |
| 4 | 4-imidazoleacetate | 0.561001295 | 1.475292782 | 0.00068275 |
| 5 | 5-oxoproline | 0.434527232 | 1.351467887 | 4.12627E−06 |
| 6 | aconitate [cis or trans] | 0.523460601 | 1.437399013 | 0.000720695 |
| 7 | adipate (C6-DC) | 0.293464336 | 1.225579725 | 0.021999483 |
| 8 | alanine | 0.463441718 | 1.378827252 | 9.22971E−06 |
| 9 | alpha-ketoglutarate | 0.223848168 | 1.167844482 | 0.126047656 |
| 10 | arabonate/xylonate | 0.544364971 | 1.45837827 | 0.042519418 |
| 11 | azelate (C9-DC) | 0.351317553 | 1.275725162 | 0.005087577 |
| 12 | beta-citryglutamate | 0.467446649 | 1.382660203 | 0.016141134 |
| 13 | choline | 0.283321874 | 1,216993847 | 0.015545954 |
| 14 | cis-urocanate | 0.639671208 | 1,557974054 | 2.33224E−06 |
| 15 | citraconate/glutaconate | 0.460210012 | 1.375742069 | 0.000116885 |
| 16 | fructose | 0.225626142 | 1.169284618 | 0.288871001 |
| 17 | fumarate | 0.34431474 | 1.269547822 | 0.027794183 |
| 18 | gamma-glutamylalanine | 0.424110429 | 1.341744923 | 0.045952367 |
| 19 | gamma-glutamylglutamine | 0.309613559 | 1.239375676 | 0.096328655 |
| 20 | gamma-glutamylglycine | 0.544635809 | 1.458652079 | 0.00281599 |
| 21 | gamma-glutamylisoleucine | 0.452863724 | 1.368754516 | 0.001564193 |
| 22 | gamma-glutamylleucine | 0.507610478 | 1,421693508 | 0.00197086 |
| 23 | gamma-glutamylserine | 0.502571602 | 1.416736644 | 0.001120818 |
| 24 | gamma-glutamylthreonine | 0.475991305 | 1.390873589 | 0.0014828 |
| 25 | gamma-glutamyltryptophane | 0.500982815 | 1.415177303 | 0.004224649 |
| 26 | gamma-glutamylvaline | 0.487642689 | 1.402151938 | 0.000258416 |
| 27 | glutarate (C5-DC) | 0.23915041 | 1.18029739 | 0.032509061 |
| 28 | glycerate | 0.26215793 | 1.199271189 | 0.031130415 |
| 29 | glycerol 3-phosphate | 0.733675298 | 1.662869899 | 0.000458764 |
| 30 | glycine | 0.454261383 | 1.370081184 | 0.00046025 |
| 31 | isovalerylglycine | 0.416550126 | 1.33473203 | 0.006928031 |
| 32 | kynurenate | 1.222806378 | 2.33400294 | 0.004630418 |
| 33 | lactate | 0.356163108 | 1.280017122 | 0.038356821 |
| 34 | linoleoyl ethanolamide | 0.460563945 | 1.376079618 | 0.02239249 |
| 35 | malate | 0.36932128 | 1,291744983 | 0.004359211 |
| 36 | maleate | 0.612820901 | 1.529246422 | 6.74611E−06 |
| 37 | malonate | 0.527575029 | 1.441504188 | 7.05402E−06 |
| 38 | maltose | 0.70983322 | 1.635615024 | 0.055872613 |
| 39 | methionine sulfoxide | 0.292698208 | 1.224929067 | 0.02369457 |
| 40 | methylsuccinate | 0.427132207 | 1.344558202 | 5.33979E−05 |
| 41 | N-acetylarginine | 0.723274866 | 1.650925326 | 0.007175595 |
| 42 | N-acetylalanine | 0.263559676 | 1.200436987 | 0.018347588 |
| 43 | N-acetylaspartate (NAA) | 0.537552815 | 1.451508294 | 0.015127349 |
| 44 | N-acetylglycine | 0.39320947 | 1.313311796 | 0.004112891 |
| 45 | N-acetylhistidine | 0.33993252 | 1.265697391 | 0.009064176 |
| 46 | N-acetylphenylalanine | 0.418235714 | 1.33629239 | 0.02762647 |
| 47 | N-acetylthreonine | 0.193190462 | 1.143289261 | 0.051522665 |
| 48 | N-acetylvaline | 0.378148188 | 1.299672553 | 0.018932275 |
| 49 | oleamide | 0.482884878 | 1.397535449 | 0.004567401 |
| 50 | ornithine | 0.400608101 | 1.320064205 | 0.0030726 |
| 51 | palmitamide (16:0) | 0.574514155 | 1.489175881 | 0.000267398 |
| 52 | pimelate (C7-DC) | 0.264673385 | 1.20136404 | 0.021751008 |
| 53 | proline | 0.386162431 | 1.306912389 | 0.000143603 |
| 54 | salicylate | 0.647492446 | 1.566443188 | 0.001580432 |
| 55 | sebacate (C10-DC) | 0.304064132 | 1.234617488 | 0.003014042 |
| 56 | serine | 0.425638239 | 1.34316658 | 6.74308E−05 |
| 57 | suberate (C8-DC) | 0.33608662 | 1.262327821 | 0.004204843 |
| 58 | succinate | 0.306667853 | 1.236847691 | 0.082821617 |
| 59 | undecanedioate (C11-DC) | 0.414292193 | 1.332644702 | 0.000297138 |
| 60 | S-amino-omegacaprolactam | 1.251717593 | 2.381247524 | 0.000567402 |
| | (isomer) | | | |

Evaluation of the Bacterial Microbiome

None of the participants received any antibiotics or systemic antifungals one month before sampling, had any severe skin disorder, and had used any skin or systemic treatments for depigmentation/whitening three months before sampling, or any exfoliating product one month before sampling.

They were asked to wash their face using a neutral soap provided without antibacterial compounds for 3 days (once a day) before sampling. The last shampoo and the last soap were applied respectively 48 and 24 h before sampling. No other product was authorized on the scalp, the hair and the face until the sampling was complete.

The sampling of microbiota was carried out in a controlled atmosphere at 22° C. and 60% humidity. The samples for the analysis of the microbiome were collected using sterile dry cotton buds that were heated to 150° C. and pre-moistened with an ST solution (0.15 M NaCl with 0.1% tween 20). For the cheek samples, the swabs were soaked in a collection buffer and rubbed firmly on the cheek for 60 seconds to cover a surface of 1 cm×2 cm. After sampling, each cotton bud was placed in a microtube and immediately frozen in liquid nitrogen, and stored at ˜80° C. before extraction of the genomic DNA (DNAg).

The profiling of the bacterial 16S rDNA was carried out as follows:
Preparation of the amplicon sample for the sequencing of the 16$ rRNA gene

the DNAg was extracted using the PowerSoil DNA® isolation kit (MO BIO Laboratories, Carlsbad, Calif., USA) by following the manufacturer's instructions with the modifications described in Leung et at (2014) Appl. Environ. Microbiol 80: 6760-6770. In addition, following elution C6, the eluate was passed through the same column filter an additional time in order to increase the yield. Negative controls of water without DNA were extracted in parallel. Each sample of DNAg was subjected to a PCR in triplicate with primers targeting the region V1-3 of the bacterial 16S rRNA, which is more precise for obtaining an image of the bacterial community of the skin (Meisel et al. (2016) J. Invest. Dermatol. 136:947-956). For the analysis of the 16S rRNA, the amplicon PCR and the indexing PCR were conducted on a PCR 7500 Fast Real-Time PCR System (Applied Biosystems, Foster City, Calif., USA), and the amplicons were purified with DNA/RNA purification beads (SeqMatic, Fremont, Calif., USA). The preparation of the library and the paired-end sequencing of the bacterial nucleic acids of 300 bp on the Illumina Miseq® platform were carried out by SeqMatic LLC (Fremont, Calif., USA).
Processing of the sequence of the rRNA 16S gene and bioinformatics analysis

The bacterial and fungal readings paired respectively in .fastq format were merged by using the “-fastq_mergeairs” command in USEARCH. The merged readings were filtered for quality control using the “-fastq_filter” command in USEARCH, with a maximum expected error rate of 0.01. The merged readings were cut at 45 bp and the shorter readings were eliminated. The filtered readings were subjected to a OTU grouping at 97% sequence identity using the UPARSE algorithm (Edgar (2013) Nature Methods 10:996-998), and the taxonomic information was provided for the sequences representative of bacterial OTUs by using the “assign_taxonomy.py” command in QIIME (version 1.9) against the SILVA database (128 outputs). The OTUs in the taxonomic lines present in more than 5% of the negative controls were considered as potential contaminants (Leung et al. (2018) Microbiome 6: 26), and were eliminated from the dataset. In addition, the chimeric, chloroplast and mitochondria OTUs were also eliminated. Following the quality control and the elimination of undesirable readings, a total of 9,656,916 bacterial readings was retained.

TABLE
F.C. bacterial microbiome, GP1 versus GP2

| | | Log2 F.C. | F.C. | |
|---|---|---|---|---|
| No. | Microbe | (GP1 vs GP2) | (GP1 vs GP2) | p value |
| 1 | Propionibactenum acnes (species) | −0.123218238 | 0.918137264 | 9.1793E−06 |
| 2 | Micrococcaceae (family) | 1.180033301 | 2.265820071 | 6.38354E−05 |
| 3 | Brachybacterium (genus) | 0.790931581 | 1.730191326 | 0.000383131 |
| 4 | Brevibacterium (genus) | 1.277515888 | 2.424212032 | 3.71944E−06 |
| 5 | Burkholderiales (order) | 0.865917746 | 1.82249865 | 1.16516E−06 |
| 6 | Paracoccus (genus) | 0.566027163 | 1.480441171 | 0.001358025 |
| 7 | Rhodobacteraceae (family) | 0.514561357 | 1.428559728 | 0.000100992 |
| 8 | Fusobacterium (genus) | −0.349593237 | 0.78480534 | 0.028291382 |
| 9 | Micrococcus luteus (species) | 0.017750243 | 1.01237953 | 0.794723678 |

Statistical Analysis

In the original scale, the measurements of metabolites are standardized in terms of raw surface counts, each metabolite is then brought to scale in order to obtain a median equal to 1 and the missing values are imputed with the minimum. The statistical analyses are conducted on transformed values.

A multi-block statistical analysis was conducted in order put into relation in the same model the metabolites, the microbiome data sampled on the cheek and the PAH data. This analysis allowed the inventors to identify relevant groups of individuals and to characterize these groups with clinical variables using approaches of X² test or variance analysis according to the type of clinical scores.

More precisely, a sparse generalized canonical correlation analysis (Witten et al. (200$) Biostatistics 10: 515-534) was conducted in order to select the descriptors significantly associated with the covariance/correlation structure between the blocks. Then, a regularized PCA-consensus, called MAXVAR-A (Tenenhaus et aL (2017) Psychometrika 82: 737-777), based on the selected descriptors, was implemented in order to construct a consensus space. Finally, a hierarchical grouping on the consensus space made it possible to reveal the two characteristic groups GP1 and GP2.

The comparisons between groups, based on the block descriptors, were carried out using Student's T test.

1. A method for diagnosing esthetic degradations of skin in a subject, comprising a step (a) of determining, in a skin sample of the subject, the level of at least one marker chosen from the group constituted of (i) bacteria that comprise a nucleic acid encoding a 16S rRNA of sequence at least 90% identical to the sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9, and (ii) metabolites of these bacteria chosen from 3-hydroxy-3-methylglutarate, 3-methylglutarate/2-methylglutarate, 4-guanidinobutanoate, 4-imidazoleacetate, 5-oxoproline, aconitrate, adipate, alanine, alpha-cetoglutarate, arabonate/xylonate, azelate, beta-citrylglutamate, choline, cis-urocanate, citraconate/glutaconate, fructose, fumarate, gamma-glutamylalanine, gamma-glutamylglutamine, gamma-glutamylglycine, gamma-glutamylisoleucine, gamma-glutamylleucine, gamma-glutamylserine, gamma-glutamylthreonine, gamma-glutamyltryptophane, gamma-glutamylvaline, glutarate, glycerate, glycerol-3-phosphate, glycine, isovalerylglycine, kynurenate, lactate, linoleoyl ethanolamide, malate, maleate, malonate, maltose, methionine sulfoxide, methylsuccinate, N-acetylalanine, N-acetylaspartate, N-acetylarginine, N-acetylglycine, N-acetylhistidine, N-acetylphenylalanine, N-acetylthreonine, N-acetylvaline, oleamide, ornithine, palmitamide, pimelate, proline, salicylate, sebacate, serine, suberate, succinate, undecanedioate and S-amino-omega caprolactam.

2. The method for diagnosing according to claim 1, wherein said at least one marker is chosen from the group constituted of (i) bacteria of the species Propionibacterim acnes, bacteria of the family Micrococcaceae, bacteria of the genus Brachybacterium, bacteria of the genus Brevibacterium, bacteria of the order Burkholderiales, bacteria of the genus Parococcus, bacteria of the family Rhodobacteraceae and bacteria of the genus Fusobacterium, and (ii) metabolites of these bacteria chosen from 3-hydroxy-3-methylglutarate, 3-methylglutarate/2-methylglutarate, 4-guanidinobutanoate, 4-imidazoleacetate, 5-oxoproline, aconitrate, adipate, alanine, alpha-cetoglutarate, arabonate/xylonate, azelate, beta-citrylglutamate, choline, cis-urocanate, citraconate/glutaconate, fructose, fumarate, gamma-glutamylalanine, gamma-glutamylglutamine, gamma-glutamylglycine, gamma-glutamylisoleucine, gamma-glutamylleucine, gamma-glutamylserine, gamma-glutamylthreonine, gamma-glutamyltryptophane, gamma-glutamylvaline, glutarate, glycerate, glycerol-3-phosphate, glycine, isovalerylglycine, kynurenate, lactate, linoleoyl ethanolamide, malate, maleate, malonate, maltose, methionine sulfoxide, methylsuccinate, N-acetylalanine, N-acetylaspartate, N-acetylarginine, N-acetylglycine, N-acetylhistidine, N-acetylphenylalanine, N-acetylthreonine, N-acetylvaline, oleamide, ornithine, palmitamide, pimelate, proline, salicylate, sebacate, serine, suberate, succinate, undecanedioate and S-amino-omega caprolactam.

3. The method for diagnosing according to claim 1, wherein said at least one marker is chosen from the group constituted of (i) bacteria of the species Micrococcus luteus and bacteria of the species Paracoccus sp., and (ii) metabolites of these bacteria chosen from kynurenate, 4-imidazoleacetate, maleate, ornithine, 4-guanidinobutanaoate, cis-urocanate, malonate, gamma-glutamylleucine, N-acetylarginine and glycerol-3-phosphate.

4. The method for diagnosing according to claim 1, the method further comprising the steps consisting of:
(b) comparing the level of said at least one marker measured in step (a) with a control, and
(c) based on the comparison of step (b), determining if the skin of the subject displays esthetic degradations.

5. The method for diagnosing according to claim 1, wherein said at least one marker is a bacterium, and the level of said at least one marker is determined by measuring the level of the corresponding 16S rRNA gene.

6. The method for diagnosing according to claim 1, wherein said at least one marker is a metabolite, and the level of said at least one marker is determined by liquid chromatography-mass spectrometry.

7. The method for diagnosing according to claim 1, wherein the skin sample is taken using a D-squame® disc.

8. The method for diagnosing according to claim 1, wherein the esthetic degradation of the skin is linked to pollution.

9. The method for diagnosing according to claim 1, wherein the esthetic degradation of the skin is selected from esthetic pigmentary disorders, lack of radiance and heterogeneity in the complexion.

10. A method of evaluating the cutaneous exposure of a subject to pollution, comprising a step (a) of determining, in a skin sample of the subject, the level of at least one marker chosen from the group constituted of (i) bacteria that comprise a nucleic acid encoding a 16S rRNA of sequence at least 90% identical to the sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9, and (ii) metabolites of these bacteria chosen from 3-hydroxy-3-methylglutarate, 3-methylglutarate/2-methylglutarate, 4-guanidinobutanoate, 4-imidazoleacetate, 5-oxoproline, aconitrate, adipate, alanine, alpha-cetoglutarate, arabonate/xylonate, azelate, beta-citrylglutamate, choline, cis-urocanate, citraconate/glutaconate, fructose, fumarate, gamma-glutamylalanine, gamma-glutamylglutamine, gamma-glutamylglycine, gamma-glutamylisoleucine, gamma-glutamylleucine, gamma-glutamylserine, gamma-glutamylthreonine, gamma-glutamyltryptophane, gamma-glutamylvaline, glutarate, glycerate, glycerol-3-phosphate, glycine, isovalerylglycine, kynurenate, lactate, linoleoyl ethanolamide, malate, maleate, malonate, maltose, methionine sulfoxide, methylsuccinate, N-acetylalanine, N-acetylaspartate, N-acetylarginine, N-acetylglycine, N-acetylhistidine, N-acetylphenylalanine, N-acetylthreonine, N-acetylvaline, oleamide, ornithine, palmitamide, pimelate, proline, salicylate, sebacate, serine, suberate, succinate, undecanedioate and S-amino-omega caprolactam.

11. The method for diagnosing according to claim 2, the method further comprising the steps consisting of:
(b) comparing the level of said at least one marker measured in step (a) with a control, and
(c) based on the comparison of step (b), determining if the skin of the subject displays esthetic degradations.

12. The method for diagnosing according to claim 3, the method further comprising the steps consisting of:
(b) comparing the level of said at least one marker measured in step (a) with a control, and
(c) based on the comparison of step (b), determining if the skin of the subject displays esthetic degradations.

13. The method for diagnosing according to claim 2, wherein said at least one marker is a bacterium, and the level of said at least one marker is determined by measuring the level of the corresponding 16S rRNA gene.

14. The method for diagnosing according to claim 3, wherein said at least one marker is a bacterium, and the level of said at least one marker is determined by measuring the level of the corresponding 16S rRNA gene.

15. The method for diagnosing according to claim 4, wherein said at least one marker is a bacterium, and the level of said at least one marker is determined by measuring the level of the corresponding 16S rRNA gene.

16. The method for diagnosing according to claim 2, wherein said at least one marker is a metabolite, and the level of said at least one marker is determined by liquid chromatography-mass spectrometry.

17. The method for diagnosing according to claim 3, wherein said at least one marker is a metabolite, and the level of said at least one marker is determined by liquid chromatography-mass spectrometry.

18. The method for diagnosing according to claim 4, wherein said at least one marker is a metabolite, and the level of said at least one marker is determined by liquid chromatography-mass spectrometry.

19. The method for diagnosing according to claim 2, wherein the esthetic degradation of the skin is linked to pollution.

20. The method for diagnosing according to claim 3, wherein the esthetic degradation of the skin is linked to pollution.


SEQUENCE LISTING (9 sequences)

SEQ ID NO: 1 - DNA, 450 bp, Propionibacterium acnes
agagtttgatcctggctcaggacgaacgctggcggcgtgcttaacacatgcaagtcgaacggaaaggccctgcttttgtggggtgctcgagtggcgaacgggtgagtaacacgtgagtaacctgcccttgactttgggataacttcaggaaactggggctaataccggataggagctcctgctgcatggtgggggttggaaagtttcggcggttggggatggactcgcggcttatcagcttgttggtggggtagtggcttaccaaggctttgacgggtagccggcctgagagggtgaccggccacattgggactgagatacggcccagactcctacgggaggcagcagtggggaatattgcacaatgggcggaagcctgatgcagcaacgccgcgtgcgggatgacggccttcgggttgtaaaccgctttcgcctgtgacgaagcgtgag

SEQ ID NO: 2 - DNA, 450 bp, Artificial Sequence
  Micrococcaceae (family) 16S rDNA
agagtttgatcctggctcaggatgaacgctggcggcgtgcttaacacatgcaagtcgaacgatgaagcccagcttgctgggtggattagtggcgaacgggtgagtaacacgtgagtaacctgcccttgactctgggataagcctgggaaactgggtctaatactggatatgacttctcaccgcatggtggggggtggaaagatttattggtcttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagccttcggg

SEQ ID NO: 3 - DNA, 450 bp, Artificial Sequence
  Brachybacterium (genus) 16S rDNA
agagtttgatcctggctcaggacgaacgctggcggcgtgcttaacacatgcaagtcgaacgatgacgacagggcttgccttgtctgattagtggcgaacgggtgagtaacacgtgagtaacctgcccccctcttcgggataacctcgggaaatcggggctaataccggatatgacttcgcaccgcatggtgtgttgtggaaagtttttcggtgggggatggactcgcggcctatcagtttgttggtgaggtaatggctcaccaaggcgatgacgggtagccggcctgagagggcgaccggccacactgggactgagacacggcccagactcctacgggaggcagcagtggggaatattgcacaatgggcgaaagcctgatgcagcgacgccgcgtgggggatgacggccttcgggttgtaaacctctttcagtagggaagaagcgaaagt

SEQ ID NO: 4 - DNA, 450 bp, Artificial Sequence
  Brevibacterium (genus) 16S rDNA
agagtttgatcctggctcaggacgaacgctggcggcgtgcttaacacatgcaagtcgaacgctgaagcctgcagcttgctgtgggtggatgagtggcgaacgggtgagtaacacgtgagtaacctgcccctgactctgggataagcccgggaaactgggtctaataccggatatgaccattctccgcatggagtggtggtggaaagtttttcggttggggatgggctcgcggcctatcagcttgttggtggggtgatggcctaccaaggcgacgacgggtagccggcctgagagggcgaccggccacactgggactgagacacggcccagactcctacgggaggcagcagtggggaatattgcacaatgggggaaaccctgatgcagcgacgccgcgtgggggatgacggccttcgggttgtaaacctctttcagtagggaagaagcctt

SEQ ID NO: 5 - DNA, 450 bp, Artificial Sequence
  Burholderiales (order) 16S rDNA
agagtttgatcctggctcagattgaacgctggcggcatgccttacacatgcaagtcgaacggcagcatgatctagcttgctagattgatggcgagtggcgaacgggtgagtaatacatcggaacgtgccctgtagtgggggataactagtcgaaagattagctaataccgcatacgacctgagggtgaaagtgggggaccgcaaggcctcatgctataggagcggccgatgtctgattagctagttggtggggtaaaggcccaccaaggcgacgatcagtagctggtctgagaggacgatcagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaatcgc

SEQ ID NO: 6 - DNA, 450 bp, Artificial Sequence
  Paracoccus (genus) 16S rDNA
agagtttgatcctggctcagaacgaacgctggcggcaggcctaacacatgcaagtcgagcgcgcccttcggggtgagcggcggacgggtgagtaacgcgtgggaacgtgcccttctctgcggaatagcctcgggaaactgagagtaataccgcatacgccctttgggggaaagatttatcggagaaggatcggcccgcgttggattaggtagttggtggggtaatggcccaccaagccgacgatccatagctggtttgagaggatgatcagccacactgggactgagacacggcccagactcctacgggaggcagcagtggggaatcttagacaatgggggcaaccctgatctagccatgccgcgtgagtgatgaaggccttagggttgtaaagctctttcagctgggaagataatgacggtaccagcagaagaagccccggctaactcc

SEQ ID NO: 7 - DNA, 450 bp, Artificial Sequence
  Rhodobacteraceae (family) 16S rDNA
agagtttgatcctggctcagaacgaacgctggcggcaggcttaacacatgcaagtcgagcgagatcttcgggtctagcggcggacgggtgagtaacgcgtgggaacgtgccctttgctacggaatagtcctgggaaactgggggtaataccgtatacgcccttttggggaaagatttatcggcgaaggatcggcccgcgttggattaggtagttggtggggtaatggcctaccaagccgacgatccatagctggtttgagaggatgatcagccacactgggactgagacacggcccagactcctacgggaggcagcagtggggaatcttagacaatgggggcaaccctgatctagccatgccgcgtgagtgatgaaggccttagggttgtaaagctctttcagctgggaagataatgacggtaccagcagaagaagccccggctaactcc

SEQ ID NO: 8 - DNA, 450 bp, Artificial Sequence
  Fusobacterium (genus) 16S rDNA
agagtttgatcctggctcaggatgaacgctgacagaatgcttaacacatgcaagtctacttgaacttcggtttgggtggcggacgggtgagtaacgcgtaaagaacttgcctcacagttagggacaacatttggaaacgaatgctaatacctgatattatgattttagggcatcctaagattatgaaagctatatgcgctgtgagagagctttgcgtcccattagctagttggagaggtaacggctcaccaaggcgatgatgggtagccggcctgagagggtgaacggccacaaggggactgagacacggcccttactcctacgggaggcagcagtggggaatattggacaatggaccaaaagtctgatccagcaattctgtgtgcacgatgacgtttttcggaatgtaaagtgctttcagttgggaagaaaaaaatgacggtaccaaca

SEQ ID NO: 9 - DNA, 450 bp, Micrococcus luteus
agagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtg